(19) United States
(12) Patent Application Publication
(10) Pub. No.: US 2014/0142487 A1
(43) Pub. Date: May 22, 2014

(54) Portable Decompression vest/belt

(71) Applicant: John Joseph Arico, Roseville, CA (US)

(72) Inventor: John Joseph Arico, Roseville, CA (US)

(21) Appl. No.: 13/364,688

(22) Filed: Nov. 19, 2012

(51) Int. Cl. A61F 5/02

(52) U.S. Cl. CPC A61F 5/02
USPC 602/36

(57) ABSTRACT

Inexpensive, portable, multi purpose decompression device for use in any and all seated positions. Vest is designed to encircle any part of torso and be lifted by strapping that connects to a vertical anchoring point for lift or decompression. Patient can exert any amount of reasonable lift to decompress as needed with their own gravitational weight. Vest will have multiple connecting points in an effort to apply specific angular direction to different types of decompression needs. The connecting points will be clipped/buckled or strapped to a vertical anchoring point that will allow the patient to adjust the amount of decompression needed by pulling the adequate amount of tension, one click at a time, from said anchoring point by pulling on the adjusting/Lifting strap.

### BACKGROUND OF THE INVENTION

1. Field of the Invention

The invention relates to the needs of individuals with compression pain and discomfort in a seated position of any kind. This is generally caused by weight or compression load on nerves and or skeletal issues specific that one's needs. It could be caused by compression load to the hips, pelvis, spine, chest or any internal point of pain or discomfort.

2. Background Art

The biggest problem with individuals with chronic or occasional discomfort in a seated position is that they still have to go on with their daily activities. Driving a motorized vehicle, sitting in a workplace or even trying to relax at home are just a few of the painful experiences within their lives. With the exception of complicated and expensive alternatives that are all prone to being in a harnessed and laying flat on their backs, there's really no items that can relieve this type of problem. Most individuals only find relief in a horizontal position. And some can't find relief without some form or traction exerted to their body in that position. This type of relief is not suited for practical day to day living.

Saunders, U.S. Pat. No. 8,083,705 discloses a low cost cervical or spinal decompression device that sits on a flat surface and allows the patient to lay flat on the unit to exert traction upon themselves. The device allows the patient to use a hand controlled pump that creates a pneumatic exertion of traction upon the flatbed and themselves as needed.

Nichols, U.S. Pat. No. 7,601,132 discloses an elaborate device that allows a patient to actually sit in a chair like position. The mechanisms attach in various ways to exert traction from above the shoulders or below. One by lifting the cervical area in traction oriented way and the other by strapping the torso of the patient to the back of the chair device and then lowering the seating platform.

Gilliam, U.S. Pat. No. 7,144,380 discloses a device that creates traction in a horizontal or vertical position. This can be done two ways; applying soft wraps to the hip area to exert traction while laying on one's back. The other traction attempt is in a sitting position but connected to a vertical point using a counter weight and pulleys. It designed more as a method.

Henke, U.S. Pat. No. 7,282,039 discloses a device that allows self adjustable lumbar traction in a supine position. The unit consists of a body harness that encircles the torso and is anchored to a support. The anchor points can be angled either from the feet area or the head area. The device works in a horizontal position in different applications. The present invention is distinguished over the prior art in general, and these patents in general by being able to be used in a horizontal fashion but specifically in a vertical seated position. It has a wrap around vest/belt the encircles the torso at a low, high or medium point and any where in between. The vest is made of any breathable material that wraps around the torso and is adjoined by hook and loop, buckles, snaps, clasps or any point of adjoinment. The vest has stretchable fibers as well as fixed fibers like piano wire meshed throughout the material for strength and give within the same material. The vest has six primary points of connection to the vertical anchor straps as well as four points of connection to the belt area of one's pants. The points of connection are located so that traction can be directed in any slight horizontal direction as well as vertical by attaching strapping to any combination of connecting points to give different angles of traction relief. The connection points are located both front and back and can be attached with any type of adjoinment connecting items. Typically buckles, straps, clasps, or any such type of connection device. This allows 10 possible connecting points on the vest that can be used in as many different angles need for most any specific traction or decompression needs. The anchor straps attach to a combination of these connecting points and then to the final anchor point to be used with the individual's gravitational tension exerted on the straps and their connecting points. The final anchor is any point above the vest connection points and allows a point of traction to take place. This vest/belt in conjunction with the upper anchoring and self gravitational traction is the foundation for the patenting aspects of this invention. It is the first of any traction oriented invention that al lows an individual to drive a car, sit in an office environment or a seated position of any kind as a temporary and pain free answer to their day-to-day living needs.

### SUMMARY OF THE INVENTION

It is therefore an object of the present invention to provide a portable back decompression vest/belt. The device will be comfortable, breathable, strong and could vary in height from a short belt like size to a full vest or any size in-between. The device will be made of a multi fibered nylon/piano wire mesh contained within a breathable material that will wrap or encompass the body's torso at many different points. It can be located in and around the waist area, abdomen, sternum or chest area. It can be attached or connected to the belt area of the individual by the connecting buckles, hook and loop, or any means of adjoinment attached to the base of the device. Once secured around the individual's torso it can be attached via buckles, straps hook and loop or any other type of attachment. Connecting points or attachment points to the top six attached buckles or clasps or any means of adjoinment will allow traction to be administered as needed by user. The attachment strap or straps will be anchored from one of many anchor points that can be used for this device.

The ability to attach to the vest/belt at any point front or back, single or double points of connection will allow different directional pull or traction upon the vest for that individual's specific needs. An example would be that a person with a bad left hip may attach to the front left ring/buckle/clasp and on the back side in an effort to balance the load to the back left point of connection. This will cause the custom strap to have a solid connection to the individual's weight and torso. The strap that is now connected to the front left and on the other side to the back left connector is now running through the tension/saw tooth buckle that is connected to an anchor above. By pulling on the adjusting strap that goes through the tension/saw tooth buckle it will create lift in small incremental adjustments that in turn creates as needed traction. This will allow individuals with compression pain whether it be spinal, hip or any area that would benefit from a slight to moderate decompression during any and all seated positions. Most notably would be in an office environment, automotive and personal use seated applications.

### BRIEF DESCRIPTION OF THE DRAWINGS

FIG. 1 is a view of the Anchor Point and the associated strapping to attach to vest/belt

FIG. 2 is a view of the actual vest/belt and its associated connections and make-up.

FIG. 3 is a stick figure showing the vest/belt in actual use in a seated position.

FIG. 4 is a view of the type of buckle being used for the actual lifting capabilities of the vest/belt to the anchoring point.

FIG. 4-a shows a side view of the buckle.

FIG. 4-b shows a front view of the buckle

FIG. 5 depicts the quick connect/quick release buckle and tensioner

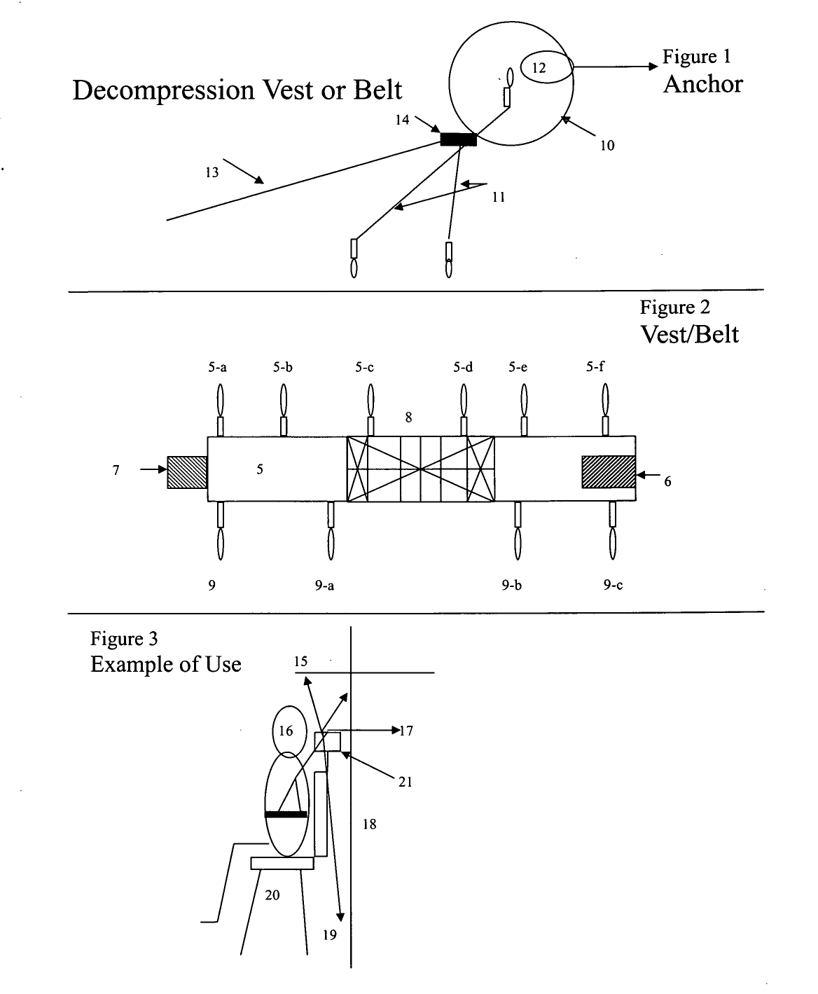

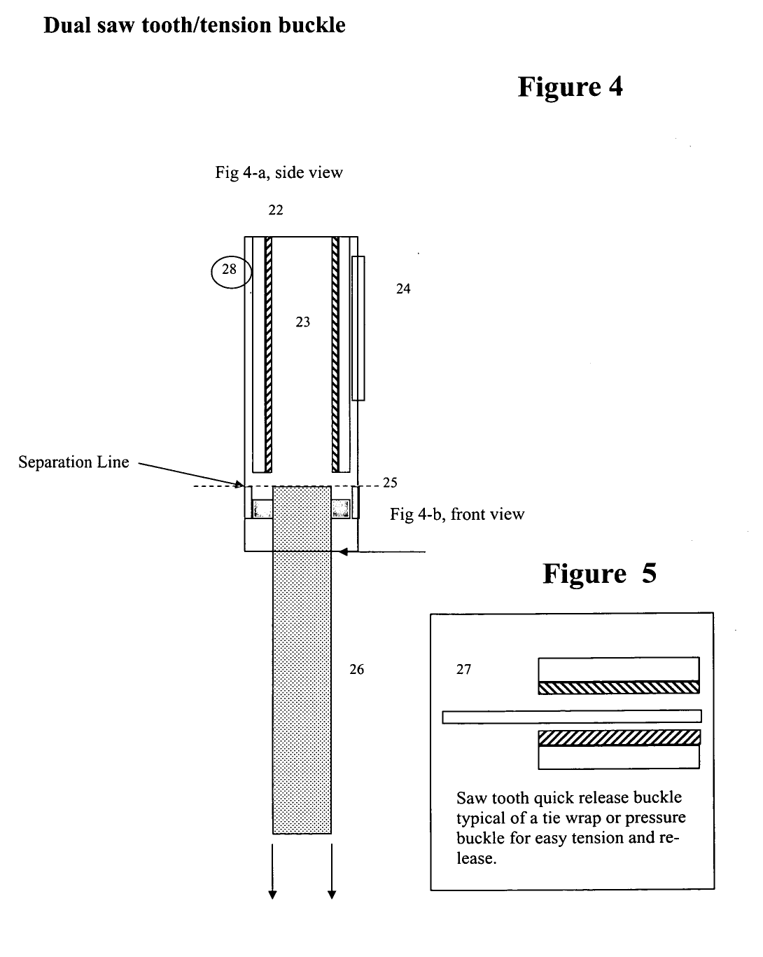

FIG. 1 shows the connection of the belt and the anchor point. Showing belt 13 running thru buckle 14 to the to loop strap or connector (10) to an anchor point 12, which in turn connects to the vest connector strap 11.

FIG. 2 pertains to the actual vest/belt. It shows a wrap around item that is made of a multi-fiber material that combines piano wire for strength and nylon lengths.

# 7 Pertains to the applying side of the hook and look connection

# 8 refers to the cross section of nylon and piano wire cross meshing for the composition of the vest/belt. This view is strictly done as an internal quick look at the material only.

# 10 references the looped strapping that is designed to encompass the point of anchor used in the lifting of one's gravitation tension on the vest itself. It is designed to be able to click or lock in small incremental tension producing strands for give and flexibility. This can be short in height or tall, it can be a vest with arm holes to place your arms through or it could be any thing in between. It shows a center portion for viewing purposes only (8) of a pattern of strands or facsimile. The vest/belt is held together by a hook and loop (6 & 7), quick release buckle or any point of adjoinment. The vest/belt shows six points of connection (5-a thru 5-f) on the upper side of it and four points of connection on the lower side (9 thru 9-c).

# 6 pertains to the receiving side of the hook and loop connection

# 11 refers to the dual straps that attach to the vest/belt upper connection points.

# 12 refers to the anchor point. It can be any point higher that the vest/belt connection points. The invention calls attention to the ability to connect to a headrest in a car or any mobile vehicle, a wall, floor/pulley assembly (that draws its amount of traction.

anchor and leverages of the back of a chair that creates a point of leverage higher than the point of connection to the vest/belt) or any connection needed to create a point of anchoring on a chair or seated position.

# 13 refers to the pull strap that allows the individual to pull one small amount at a time through the cross toothed buckle 14 as need to exert the proper

# 14 refers to the actual buckle that is used to allow the three straps needed for connection to the anchor, vest/belt and the individuals adjusting capabilities.

# 15 refers to the ceiling or horizontal plane or any other anchoring point avail able to create a point of connection that can be used for strapping a buckle combination needed for traction capabilities. (a fixed point)

# 16 refers to the stick figure drawing of an individual sitting in a seated environment 20 and encircled by a vest/belt in a traction type of environment.

# 17 refers to the straps that connect to the anchors 15 above the height of the vest/belt.

# 18 refers to a wall or vertical plain that can secure an anchor for a point of connection.

# 19 refers to a point of anchoring that is below the level of the vest but by applying a set of straps or pulley from the back of a chair 20 or headrest 21; one could still get a point higher than the vest/belt and allow a proper traction to occur.

# 20 refers to the actual seating area and rear backrest that comprise a chair or any version of a seated chair like environment.

# 21 refers to the headrest used in a motor vehicle environment. A point of adjoining within most vehicles across all uses.

# 22 Shows the top of the buckle assembly.

# 23 refers to a side view of the buckle assembly to show the saw tooth channel for a strap/belt to ride through.

# 24 refers to the quick release lever on the buckle assembly.

# 25 refers to the separation line between the side view of the upper portion and the front view of the lower portion of the buckle assembly that the actual belt rides on.

# 26 refers to the belt/strapping that is used throughout this buckle assembly.

# 27 refers to a brief drawing to better understand the belt/strapping going thru the saw tooth type of buckle

# 28 Typical buckle connection where a nylon belt 11 is stationary to the buckle and 13 is drawn thru the buckle with opposing tension.

### DESCRIPTION OF THE PREFERRED EMBODIMENTS

Referring to the drawings by numerals of reference, there is shown in FIG. 1, 2, 3, 4 a back decompression vest/belt in accordance with the present invention. The decompression vest 5 has a shape and look of a kidney belt, girdle, or wrap-around vest/belt. Within this design and look are twelve points of connection. Numerals 5-a, 5-b, 5-c, 5-d, 5-e, 5-f are all top mounted points of connection to the strapping/belts 11 that are connected or part of the strapping/belts 10 and 13. With 10 being a loop within the strapping or possibly a buckle, clasp or point of connection that firmly attaches to said anchor and is pulled taught by strap ping controlled by the individual applying the needed traction as they see fit using their own body weight via 11 and 13, straps/belts that have several quick release clasps for additional safety, being run through custom buckle 14 which is connected to 12 the anchor. This anchor will be the headrest in a car, truck or moving vehicle. The backrest on an office chair or recreational chair in one's house or any seated position necessary for one's day to day living conditions and comfort.

The vest/belt 5 is wrapped firmly around one's torso at any one of many heights as needed by the user. It will fit firmly around one's torso by hook and loop, clasps, buckles or any point of adjoinment 6 and 7. It has four 9, 9-a, 9-b, 9-c points of connection that hang off the lower portion of said vest/belt in an effort or desire to attach to one's personal pant belt. The vest which is connected to any one of several anchoring points 15 the ceiling, 18 the wall, 21 the head rest, 19 over the back of the back rest of the chair to floor or base or any point of connection available. The numerals 16 represent a crude figure of a person in a seated position in a chair 20. As you can see the person 16 has a vest/belt around their waste that is being pulled in a vertical direction to a point of anchor 15,18,19,21 and showing a self controlled, portable strap/belt assembly that can be used in multiple applications. The vest/belt can be raised and snugged up by pulling apart the hook and loop connectors or connectors and wrap around a higher point needed for traction to most any part of the skeletal torso area. The custom buckle 23 has a saw tooth channel that the strapping/belt runs through to create a snug fitting connection between the vest/belt applied to the torso and an anchor from above, 22. On one side of the buckle 23 there is a permanent fitting that the strapping/belt is connected to 28 with the other end looped around the anchor and run thru the channel or saw tooth area of the buckle.

The other side of the actual buckle is equipped with a quick disconnect lever to release the belt assembly completely from the lift exerted by the anchor and adjustor belt pull 13. The broken line in reflected by numeral 25 changes the view of the drawing from a side view above to a front view below showing a pin or rod that allows a strap/belt to loop over and stay connected to said buckle 23 connecting to vest/belt 5 in what ever fashion needed by the user. The numeral 27 represents the strapping/belts that ride thru the saw tooth channel in the open position before closing the channel and pulling the strap/belt thru the one-way connection.

What is claimed is:

1. A vest/belt or any facsimile that is low cost, light weight, portable mid to upper skeletal traction device designed to use one's own weight as a point of traction.
a. As stated in claim one, the device is a light weight vest/belt or any facsimile made of a combination of piano wire for strength and nylon fibers for flexibility and distribution of weight.
b As stated in claim one, device has ten points of connection in an effort to exert just the right angle on most any point of traction needed.

2. Said traction device has a custom buckle assembly for containing the strapping/belts necessary for connection, pressure and quick release.
a. As stated in claim two, device has a belt configuration for the three point connection to anchor position.

3. Said traction device will be used in a mobile vehicle anchored/suspended from the headrest of said vehicle or any other point of connection.
a. Said traction device will be anchored/suspended from wall, ceiling or floor anchor locations or any other point of connection.
b. Said traction device can be anchored over the back of a chair and anchored to the legs, rear seat assembly or bolts in a mobile vehicle or any other point of connection.
c. Said traction device is designed to be adjusted and controlled by it's user's own strength and needs.
d. Said traction device is designed to be used in the office and workplace environment.
e. Said traction device is designed to be used in the home and living environment of it's users.
f. Said traction device is designed to be anchored over the back of any chair or anchored to the back of the seat, legs or bolts in a mobile vehicle environment.
